(19) United States
(12) Patent Application Publication
(10) Pub. No.: US 2015/0126878 A1
(43) Pub. Date: May 7, 2015

(54) HEART FAILURE EVENT DETECTION AND RISK STRATIFICATION USING HEART SOUND

(71) Applicant: Cardiac Pacemakers, Inc., St. Paul, MN (US)

(72) Inventors: Qi An, Blaine, MN (US); Yi Zhang, Plymouth, MN (US); Viktoria A. Averina, Roseville, MN (US); Kenneth C. Beck, Liberty, UT (US); Pramodsingh Hirasingh Thakur, Woodbury, MN (US)

(21) Appl. No.: 14/510,498

(22) Filed: Oct. 9, 2014

Related U.S. Application Data
(60) Provisional application No. 61/899,653, filed on Nov. 4, 2013.

(51) Int. Cl. A61B 7/04; A61B 5/0205; A61B 5/00; A61B 5/01; A61B 5/11; A61B 5/0456; A61B 5/08

(52) U.S. Cl. CPC A61B 7/04; A61B 5/0456; A61B 5/0205; A61B 5/08; A61B 5/01; A61B 5/1118; A61B 5/4809
USPC 600/484; 600/528; 600/513; 600/483

(57) ABSTRACT

Devices and methods for detecting heart failure (HF) events or identifying patient at elevated risk of developing future HF events are described. A medical device can detect contextual condition associated with a patient, such as an environmental context or a physiologic context, sense a heart sound signal, and perform multiple measurements of heart sound features in response to the detected patient contextual condition meeting specified criterion. The contextual condition includes information correlating to or indicative of a change in metabolic demand of a patient. The medical device can use the physiologic signals to calculate one or more signal metrics indicative of diastolic function of the heart such as a trend of the heart sound features. The medical device can use the signal metrics to detect an HF event or to predict the likelihood of the patient later developing an HF event.

### CLAIM OF PRIORITY

This application claims the benefit of priority under 35 U.S.C. §119(e) of U.S. Provisional Patent Application Ser. No. 61/899,653, filed on Nov. 4, 2013, which is herein incorporated by reference in its entirety.

### TECHNICAL FIELD

This document relates generally to medical devices, and more particularly, to systems, devices and methods for detecting and monitoring heart failure decompensation.

### BACKGROUND

Congestive heart failure (CHF) is a major health problem and affects over five million people in the United States alone. CHF is the loss of pumping power of the heart, resulting in the inability to deliver enough blood to meet the demands of peripheral tissues. CHF patients typically have enlarged heart with weakened cardiac muscles, resulting in reduced contractility and poor cardiac output of blood.

CHF is usually a chronic condition, but can occur suddenly. It can affect the left heart, right heart or both sides of the heart. If CHF affects the left ventricle, signals that control the left ventricular contraction are delayed, and the left and right ventricles do not contract simultaneously. Non-simultaneous contractions of the left and right ventricles further decrease the pumping efficiency of the heart.

### OVERVIEW

Frequent monitoring of CHF patients and timely detection of events indicative of heart failure (HF) decompensation status can help prevent worsening of HF in CHF patients, hence reducing cost associated with HF hospitalization. Additionally, identification of patient at an elevated risk of developing future HF events such as worsening of HF can help ensure timely treatment, thereby improving the prognosis and patient outcome. Identifying and safely managing the patients having risk of future HF events can avoid unnecessary medical intervention and reduce healthcare cost.

Ambulatory medical devices can be used for monitoring HF patient and detecting HF decompensation events. Examples of such ambulatory medical devices can include implantable medical devices (IMD), subcutaneous medical devices, wearable medical devices or other external medical devices. The ambulatory or implantable medical devices can include physiologic sensors which can be configured to sense electrical activity and mechanical function of the heart, or physical or physiological variables associated with the signs and symptoms of worsening of HF. The medical device can optionally deliver therapy such as electrical stimulation pulses to a target area, such as to restore or improve the cardiac function or neural function. Some of these devices can provide diagnostic features, such as using transthoracic impedance or other sensor signals. For example, fluid accumulation in the lungs decreases the transthoracic impedance due to the lower resistivity of the fluid than air in the lungs. Fluid accumulation in the lungs can also irritate the pulmonary system and leads to decrease in tidal volume and increase in respiratory rate.

Some ambulatory medical devices can include a physiologic sensor for detecting heart sounds. Heart sounds are associated with mechanical vibrations from activity of a patient's heart and the flow of blood through the heart. Heart sounds recur with each cardiac cycle and are separated and classified according to the activity associated with the vibration. The first heart sound (S1) is associated with the vibrational sound made by the heart during tensing of the mitral valve. The second heart sound (S2) marks the beginning of diastole. The third heart sound (S3) and fourth heart sound (S4) are related to filling pressures of the left ventricle during diastole. Heart sounds are useful indications of proper or improper functioning of a patient's heart. The fluid accumulation in the lungs in HF patients can result in an elevation of ventricular filling pressure, resulting in a louder S3 heart sound.

Worsening of HF status can deteriorate diastolic function of the heart. Monitoring and trending the S3 heart sound can be useful in assessing the diastolic function of the heart and the progression of HF status. Accurate and timely detection of worsening of HF such as HF decompensation events, or reliable prediction of the risk of a patient developing future HF decompensation event may require reliable sensing of S3 heart sound and determination of the trend of S3 strength as a function of time. However, S3 is generally a weak signal compared to S1 or S2 heart sounds. S3 can be contaminated by noise or other interferences, or be affected by various physiologic or environmental conditions. As such, the reliability of the detected S3 strength or the trend of the S3 can be compromised, a S3 heart sound based HF decompensation detection algorithm may produce false positive or false negative detections, and a S3 heart sound based HF risk stratification algorithms can provide less desirable prediction performance. Additionally, the present inventors have recognized that heart sound components, such as S3 heart sound, can be more reflective of the progression of HF when the patient is under certain conditions. For example, when the patient is under elevated metabolic demand, such as when the patient is awake, or in an upright position, or under stress, the underlying HF disease or worsening of HF status can be more likely triggered and presented, and thereby sensed by physiologic sensors such as a heart sound detector. Therefore, there remains a considerable need of systems and methods that can reliably and accurately detect and trend the S3 heart sound for the use in detecting worsening of HF or identifying CHF patients with elevated risk of developing future events of worsening of HF.

Various embodiments described herein can help improve detection of an HF event indicative of worsening of HF, or improve process of identifying patients at elevated risk of developing future HF events. For example, a system can comprise an ambulatory medical device (such as an implantable medical device or a wearable medical device) that can detect an HF event or predict the risk of HF event using one or more signal metrics generated from physiologic signals. The medical device can include a context detector circuit that can detect contextual condition associated with a patient, such as an environmental context or a physiologic context of a patient. The contextual condition can include information correlating to or indicative of changes in metabolic demand of the patient, such as an elevated metabolic demand. A heart sound analyzer circuit can sense a heart sound signal, generate one or more heart sound features, and perform multiple measurements of the heart sound features in response to the detected patient contextual condition meeting specified criterion. A target event indicator generator circuit can calculate one more signal metrics indicative of diastolic function of the heart, where the signal metrics can include a trend of the heart sound features. The medical device can include a physiologic event detector circuit that can detect an HF event using the signal metrics. Additionally or alternatively, the system can include a risk stratifier circuit that can calculate a composite risk indicator indicative of the likelihood of the patient developing a future event indicative of worsening of HF.

A method can include detecting from a patient a contextual condition including environmental or physiologic context. The method includes sensing from a patient a physiologic signal indicative of heart sound (HS) and processing the heart sound signal to generate one or more heart sound features. The method includes measuring multiple measurements of the heart sound features in response to the detected patient contextual condition meeting a specified criterion, and calculating one or more signal metrics indicative of systolic function of the heart using the heart sound measurements, where the signal metrics can include a trend of the heart sound features. The method can further include using the signal metrics to detect a target physiologic event indicative of worsening of HF, or to generate a composite risk indicator that can predict the risk of the patient developing a future event indicative of worsening of HF.

This Overview is an overview of some of the teachings of the present application and not intended to be an exclusive or exhaustive treatment of the present subject matter. Further details about the present subject matter are found in the detailed description and appended claims. Other aspects of the invention will be apparent to persons skilled in the art upon reading and understanding the following detailed description and viewing the drawings that form a part thereof, each of which are not to be taken in a limiting sense. The scope of the present invention is defined by the appended claims and their legal equivalents.

### BRIEF DESCRIPTION OF THE DRAWINGS

Various embodiments are illustrated by way of example in the figures of the accompanying drawings. Such embodiments are demonstrative and not intended to be exhaustive or exclusive embodiments of the present subject matter.

FIG. 1 illustrates an example of a cardiac rhythm management (CRM) system and portions of the environment in which the CRM system operates.

FIG. 2 illustrates an example of a heart sound-based physiologic event detector circuit.

FIG. 3 illustrates an example of a heart sound-based heart failure risk stratifier circuit.

FIG. 4 illustrates an example of a context receiver circuit.

FIG. 5 illustrates an example of a HF risk stratifier/event detector circuit.

FIG. 6 illustrates examples of receiver operating characteristics (ROC) curves corresponding to representative S3 heart sound strength calculated from heart sound signals acquired during different time of day.

FIG. 7 illustrates an example of a method for evaluating cardiac diastolic function of a patient.

FIG. 8 illustrates an example of a method for detecting an HF event indicative of HF decompensation or providing a risk stratification of a future HF event.

### DETAILED DESCRIPTION

Disclosed herein are systems, devices, and methods for detecting an event indicative of worsening of HF such as an HF decompensation event, and/or for identifying patients with elevated risk of developing future events related to worsening of HF. The HF event detection or HF risk stratification can be performed using the physiologic signals such as sensed from a physiologic sensor associated with an ambulatory medical device such as an implantable cardiac device. The present inventors have recognized that contextual conditions, including ambient environmental contexts and patient physiologic contexts, can affect certain types of sensor signals in HF patients including those indicative of diastolic function of the heart. Therefore, by selectively acquiring sensor signals according to the patient contextual conditions and analyzing the signal metrics derived from the selectively acquired sensor signals, the present document can provide a method and device to detect the HF event indicative of worsening of HF, or to predict the risk of future HF event, thereby allowing immediate medical attention to the patient.

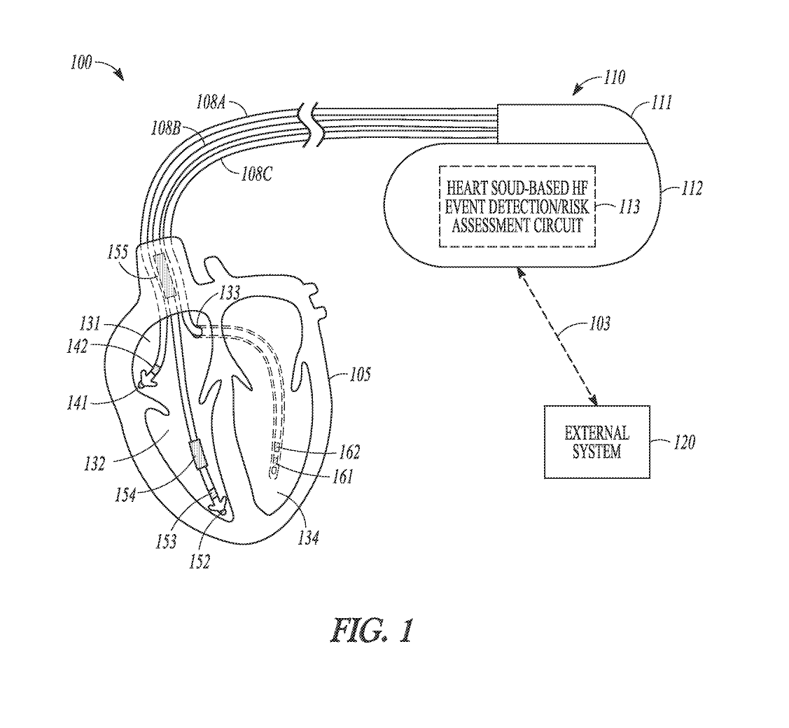

FIG. 1 illustrates an example of a Cardiac Rhythm Management (CRM) system 100 and portions of an environment in which the CRM system 100 can operate. The CRM system 100 can include an ambulatory medical device, such as an implantable medical device (IMD) 110 that can be electrically coupled to a heart 105 such as through one or more leads 108A-C, and an external system 120 that can communicate with the IMD 110 such as via a communication link 103. The IMD 110 may include an implantable cardiac device such as a pacemaker, an implantable cardioverter-defibrillator (ICD), or a cardiac resynchronization therapy defibrillator (CRT-D). The IMD 110 can include one or more monitoring or therapeutic devices such as a subcutaneously implanted device, a wearable external device, a neural stimulator, a drug delivery device, a biological therapy device, a diagnostic device, or one or more other ambulatory medical devices. The IMD 110 may be coupled to, or may be substituted by a monitoring medical device such as a bedside or other external monitor.

As illustrated in FIG. 1, the IMD 110 can include a hermetically sealed can 112 that can house an electronic circuit that can sense a physiological signal in the heart 105 and can deliver one or more therapeutic electrical pulses to a target region, such as in the heart, such as through one or more leads 108A-C. The CRM system 100 can include only one lead such as 108B, or can include two leads such as 108A and 108B.

The lead 108A can include a proximal end that can be configured to be connected to IMD 110 and a distal end that can be configured to be placed at a target location such as in the right atrium (RA) 131 of the heart 105. The lead 108A can have a first pacing-sensing electrode 141 that can be located at or near its distal end, and a second pacing-sensing electrode 142 that can be located at or near the electrode 141. The electrodes 141 and 142 can be electrically connected to the IMD 110 such as via separate conductors in the lead 108A, such as to allow for sensing of the right atrial activity and optional delivery of atrial pacing pulses. The lead 108B can be a defibrillation lead that can include a proximal end that can be connected to IMD 110 and a distal end that can be placed at a target location such as in the right ventricle (RV) 132 of heart 105. The lead 108B can have a first pacing-sensing electrode 152 that can be located at distal end, a second pacing-sensing electrode 153 that can be located near the electrode 152, a first defibrillation coil electrode 154 that can be located near the electrode 153, and a second defibrillation coil electrode 155 that can be located at a distance from the distal end such as for superior vena cava (SVC) placement. The electrodes 152 through 155 can be electrically connected to the IMD 110 such as via separate conductors in the lead 108B. The electrodes 152 and 153 can allow for sensing of a ventricular electrogram and can optionally allow delivery of one or more ventricular pacing pulses, and electrodes 154 and 155 can allow for delivery of one or more ventricular cardioversion/defibrillation pulses. In an example, the lead 108B can include only three electrodes 152, 154 and 155. The electrodes 152 and 154 can be used for sensing or delivery of one or more ventricular pacing pulses, and the electrodes 154 and 155 can be used for delivery of one or more ventricular cardioversion or defibrillation pulses. The lead 108C can include a proximal end that can be connected to the IMD 110 and a distal end that can be configured to be placed at a target location such as in a left ventricle (LV) 134 of the heart 105. The lead 108C may be implanted through the coronary sinus 133 and may be placed in a coronary vein over the LV such as to allow for delivery of one or more pacing pulses to the LV. The lead 108C can include an electrode 161 that can be located at a distal end of the lead 108C and another electrode 162 that can be located near the electrode 161. The electrodes 161 and 162 can be electrically connected to the IMD 110 such as via separate conductors in the lead 108C such as to allow for sensing of the LV electrogram and optionally allow delivery of one or more resynchronization pacing pulses from the LV.

The IMD 110 can include an electronic circuit that can sense a physiological signal. The physiological signal can include an electrogram or a signal representing mechanical function of the heart 105. The hermetically sealed can 112 may function as an electrode such as for sensing or pulse delivery. For example, an electrode from one or more of the leads 108A-C may be used together with the can 112 such as for unipolar sensing of an electrogram or for delivering one or more pacing pulses. A defibrillation electrode from the lead 108B may be used together with the can 112 such as for delivering one or more cardioversion/defibrillation pulses. In an example, the IMD 110 can sense impedance such as between electrodes located on one or more of the leads 108A-C or the can 112. The IMD 110 can be configured to inject current between a pair of electrodes, sense the resultant voltage between the same or different pair of electrodes, and determine impedance using Ohm's Law. The impedance can be sensed in a bipolar configuration in which the same pair of electrodes can be used for injecting current and sensing voltage, a tripolar configuration in which the pair of electrodes for current injection and the pair of electrodes for voltage sensing can share a common electrode, or tetrapolar configuration in which the electrodes used for current injection can be distinct from the electrodes used for voltage sensing. In an example, the IMD 110 can be configured to inject current between an electrode on the RV lead 108B and the can housing 112, and to sense the resultant voltage between the same electrodes or between a different electrode on the RV lead 108B and the can housing 112. A physiologic signal can be sensed from one or more physiological sensors that can be integrated within the IMD 110. The IMD 110 can also be configured to sense a physiological signal from one or more external physiologic sensors or one or more external electrodes that can be coupled to the IMD 110. Examples of the physiological signal can include one or more of electrocardiogram, intracardiac electrogram, arrhythmia, heart rate, heart rate variability, intrathoracic impedance, intracardiac impedance, arterial pressure, pulmonary artery pressure, left atrial pressure, RV pressure, LV coronary pressure, coronary blood temperature, blood oxygen saturation, one or more heart sounds, physical activity or exertion level, physiologic response to activity, posture, respiration, body weight, or body temperature.

The arrangement and functions of these leads and electrodes are described above by way of example and not by way of limitation. Depending on the need of the patient and the capability of the implantable device, other arrangements and uses of these leads and electrodes are possible.

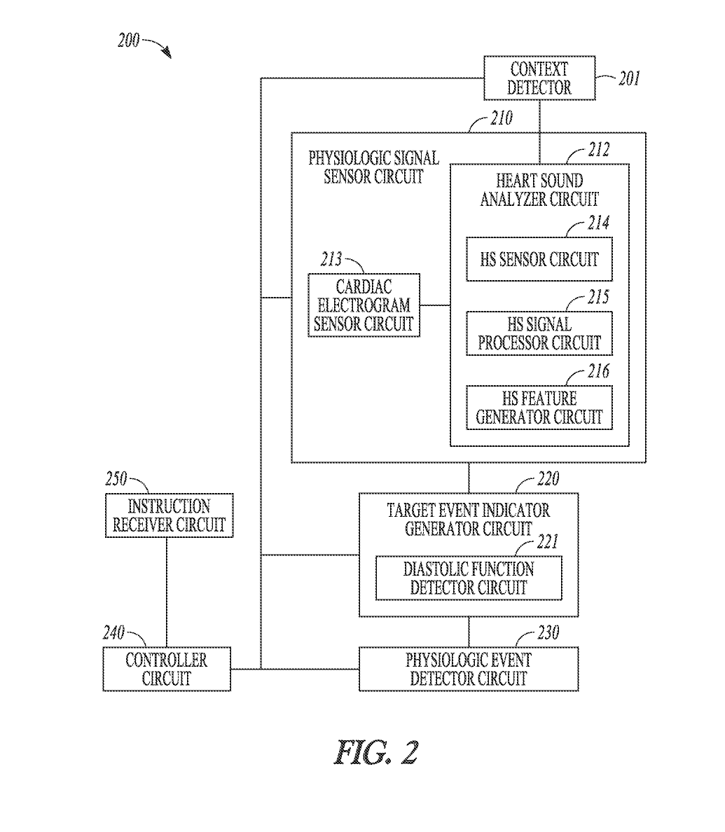

As illustrated, the CRM system 100 can include a heart sound-based HF event detection/risk assessment circuit 113. The heart sound-based HF event detection/risk assessment circuit 113 can include a physiologic signal receiver circuit, a target event indicator generator circuit, and a physiologic event detector or risk stratifier circuit. The physiologic signal receiver circuit can receive an electrogram indicative of electrical activity of the heart, and generate electrogram features using the electrogram. The electrogram can be sensed using ambulatory physiologic sensors deployed on or within the patient and communicated with the IMD 110, such as electrodes on one or more of the leads 108A-C and the can 112, or ambulatory physiologic sensors deployed on or within the patient and communicated with the IMD 110. The physiologic signal receiver circuit can include a heart sound analyzer circuit that can receive heart sound such as using ambulatory sensors for sensing a signal indicative of heart sound, and generate one or more heart sound features. The target event generator circuit can generate a signal metric indicative of cardiac diastolic function, such as S3 heart sound strength. The physiologic event detector or risk stratifier circuit can generate a trend of the detection metric for use to detect an event of worsening of HF, or to generate a composite risk indicator (CRI) indicative of the likelihood of the patient later developing an event of worsening of HF. The HF decompensation event can include one or more early precursors of an HF decompensation episode, or an event indicative of HF progression such as recovery or worsening of HF status. Examples of heart sound-based HF event detection/risk assessment circuit 113 are described below, such as with reference to FIGS. 2-5.

The external system 120 can allow for programming of the IMD 110 and can receive information about one or more signals acquired by IMD 110, such as can be received via a communication link 103. The external system 120 can include a local external IMD programmer. The external system 120 can include a remote patient management system that can monitor patient status or adjust one or more therapies such as from a remote location.

The communication link 103 can include one or more of an inductive telemetry link, a radio-frequency telemetry link, or a telecommunication link, such as an internet connection. The communication link 103 can provide for data transmission between the IMD 110 and the external system 120. The transmitted data can include, for example, real-time physiological data acquired by the IMD 110, physiological data acquired by and stored in the IMD 110, therapy history data or data indicating IMD operational status stored in the IMD 110, one or more programming instructions to the IMD 110 such as to configure the IMD 110 to perform one or more actions that can include physiological data acquisition such as using programmably specifiable sensing electrodes and configuration, device self-diagnostic test, or delivery of one or more therapies.

The heart sound-based HF event detection/risk assessment circuit 113 may be implemented at the external system 120, which can be configured to perform HF risk stratification or HF event detection, such as using data extracted from the IMD 110 or data stored in a memory within the external system 120. Portions of heart sound-based HF event detection/risk assessment circuit 113 may be distributed between the IMD 110 and the external system 120.

Portions of the IMD 110 or the external system 120 can be implemented using hardware, software, or any combination of hardware and software. Portions of the IMD 110 or the external system 120 may be implemented using an application-specific circuit that can be constructed or configured to perform one or more particular functions, or can be implemented using a general-purpose circuit that can be programmed or otherwise configured to perform one or more particular functions. Such a general-purpose circuit can include a microprocessor or a portion thereof, a microcontroller or a portion thereof, or a programmable logic circuit, or a portion thereof. For example, a “comparator” can include, among other things, an electronic circuit comparator that can be constructed to perform the specific function of a comparison between two signals or the comparator can be implemented as a portion of a general-purpose circuit that can be driven by a code instructing a portion of the general-purpose circuit to perform a comparison between the two signals. While described with reference to the IMD 110, the CRM system 100 could include a subcutaneous medical device (e.g., subcutaneous ICD, subcutaneous diagnostic device), wearable medical devices (e.g., patch based sensing device), or other external medical devices.

FIG. 2 illustrates an example of a heart sound-based physiologic event detector circuit 200, which can be an embodiment of the heart sound-based HF event detection/risk assessment circuit 113. The heart sound-based physiologic event detector circuit 200 can also be implemented in an external system such as a patient monitor configured for providing the patient's diagnostic information to an end-user. The heart sound-based physiologic event detector circuit 200 can include one or more of a context detector 201, a physiologic signal receiver circuit 210, a target event indicator generator circuit 220, a physiologic event detector circuit 230, a controller circuit 240, and an instruction receiver circuit 250.

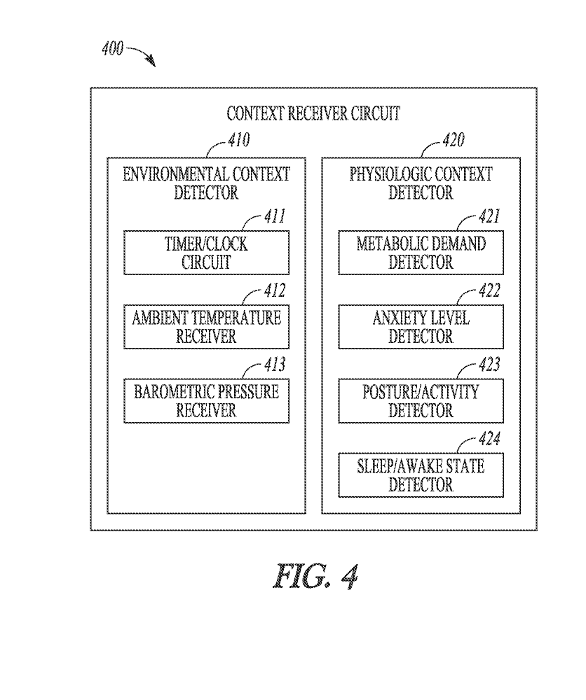

The context detector 201 can be configured to detect contextual condition associated with a patient. The contextual condition can include a patient's physiologic context or an environmental context. The physiologic context includes body-related contextual information, such as the posture, activity level, sleep or awake state, mental or emotional state, metabolic demand, body temperature, patient weight, body fluid status, and other parameters indicative of the patient's body conditions or health status. The environmental context can include factors external to the patient but likely to affect patient's health or disease states. Examples of the environmental context can include the ambient temperature, barometric pressure, humidity, or social environment. The physiologic context or the environment context can correlate to or be indicative of a change in the patient's metabolic demand such as elevated metabolic demand. Details of the context detector 201 are discussed below, such as with reference to FIG. 4.

The physiologic signal receiver circuit 210 can be configured to sense one or more physiological signals that can be indicative of worsening of HF status. The physiologic signals can be sensed using one or more physiologic sensors associated with the patient. Examples of such a physiological signal can include one or more electrograms sensed from the electrodes on one or more of the leads 108A-C or the can 112, heart rate, heart rate variability, electrocardiogram, arrhythmia, intrathoracic impedance, intracardiac impedance, arterial pressure, pulmonary artery pressure, left atrial pressure, RV pressure, LV coronary pressure, coronary blood temperature, blood oxygen saturation, one or more heart sounds, physiologic response to activity, apnea hypopnea index, one or more respiration signals such as a respiration rate signal or a tidal volume signal. In some examples, the physiologic signals can be acquired from a patient and stored in a storage device such as an electronic medical record (EMR) system. The physiologic signal receiver circuit 310 can be coupled to the storage device and retrieve from the storage device one or more physiologic signals in response to a command signal. The command signal can be issued by a system user (e.g., a physician) such as via an input device coupled to the instruction receiver 250, or generated automatically by the system in response to a specified event.

The physiologic signal sensing circuit 210 can include sub-circuits that perform signal analysis (e.g., signal amplification, digitization, or filtering) and signal feature extraction, including signal mean, median, or other central tendency measures; a histogram of the signal intensity; one or more signal trends over time; one or more signal morphological descriptors; or signal power spectral density at a specified frequency range.

As illustrated in FIG. 2, the physiologic signal sensing circuit 210 can include a heart sound analyzer circuit 212. Additionally and optionally, the physiologic signal sensing circuit 210 can include one or more other physiologic sensors, such as an electrogram sensor circuit 213, configured to generate physiologic signals to be used together with the heart sound signals in detecting the target physiologic event.

The heart sound analyzer circuit 212 can include a heart sound (HS) sensor circuit 214, a HS signal processor circuit 215, and a HS feature generator circuit 216. The HS sensor circuit 214 can be coupled to a heart sound sensor that can detect the heart sound or other forms of signals generated as a result of mechanical activities of the heart such as contraction and relaxation. Examples of the HS sensors can include an ambulatory accelerometer or an ambulatory microphone. The heart sound sensor can be external to the patient or implanted inside the body. In an example, the heart sound sensor can be within an ambulatory medical device such as the IMD 110.

The HS signal processor circuit 215 can be configured to process the HS signal including amplification, digitization, filtering, or other signal conditioning operations. In an example, the HS signal processor circuit 215 can include one or more signal filters that can filter the sensed HS signal to a specified frequency range. For example, the HS signal processor circuit 215 can include a bandpass filter adapted to filter the HS signal to a frequency range of approximately between 5 and 90 Hz. In another example, the HS signal processor circuit 215 includes a bandpass filter adapted to filter the HS signal to a frequency range of approximately between 9 and 90 Hz. In an example, the HS signal processor 215 includes a double or higher-order differentiator configured to calculate a double or higher-order differentiation of the sensed heart sound signal.

The HS feature generator circuit 216 can be configured to generate one or more heart sound features using the processed HS signal. Examples of the HS features can include timing of at least one of S1, S2 or S3 heart sounds. The HS feature generator 216 can include a HS detection window generator that can determine respective time windows for one or more HS features. The time windows can be determined with reference to a physiologic event such as R wave, Q wave, or QRS complexes detected from an electrogram such as produced by the cardiac electrogram sensor circuit 213. For example, an S1 detection window can begin at 50 milliseconds (msec) following a detected R wave and have a duration of 300 msec. An S2 detection window can begin at specified offset following a detected R wave or S1 heart sound. An S3 detection window can be determined using at least one cardiac signal feature such as the R-wave timing or the timing of S2 heart sound. The S3 detection window can have a specified duration and can begin at a specified offset following the detected S2. In an example, the offset can be 125 msec, and the S3 window duration can be 125 msec. The offset or the S3 window duration can be a function of a physiologic variable such as a heart rate. For example, the offset can be inversely proportional to the heart rate, such that the S3 detection window can start at a smaller offset following the S2 at a higher heart rate.

The HS feature generator circuit 216 can be configured to generate a HS feature from at least a portion of the HS signal within the respective HS detection window. In an example, the HS feature generator circuit 216 can calculate HS signal energy within the S3 detection window (E_{S3Win}), and detects the presence of S3 heart sound in response to the E_{S3Win} exceeds a specified threshold. In an example, the HS feature generator circuit 216 can detect the HS features adaptively by tracking the temporal locations of the previously detected HS features. For example, an S3 heart sound can be detected by adaptively tracking the timing of historically detected S3 heart sounds. A dynamic programming algorithm can be used to detect and track the S3 heart sound within the S3 detection window, such as that disclosed in the commonly assigned Patangay et al. U.S. Pat. No. 7,853,327 entitled “HEART SOUND TRACKING SYSTEM AND METHOD,” which is hereby incorporated by reference in its entirety. The HS feature generator circuit 216 can be configured to measure the S3 strength if S3 is deemed detected, such as by determining the amplitude in the time-domain HS signal, a transformed HS signal such as integrated HS energy signal, or in a frequency-domain HS signal such as the peak value of the power spectral density. In some examples, the HS feature generator circuit 216 can be configured to measure the S3 strength as the peak value of a generic measurement within the S3 detection window, such as peak envelop signal or root-mean-squared value of the portion of the HS signal within the S3 detection window.

As illustrated in FIG. 2, the heart sound analyzer circuit 212 can be coupled to the context detector 201 and receive the detected contextual conditions such as patient physiologic context or environmental context information. The HS feature generator circuit 216 can perform a plurality of HS measurements of the one or more HS features in response to the detected contextual condition meeting a specified criterion. The patient physiologic context information, or the ambient environment context, may affect the quality of the HS signals or introduce confounding factors into the HS features generated by the HS analyzer circuit 212, thereby reducing the reliability and accuracy of the HS-based physiologic event detection. Therefore, performing HS sensing or generating HS feature in accordance with specified context may help improve the reliability and accuracy of the detection of the target physiologic events. In an example, the heart sound analyzer circuit 212 can perform HS detection and feature generation in response to the context detector 201 detecting one or more contextual conditions correlating to or indicative of a change of the patient's metabolic demand, such as an elevated metabolic demand that is indicated by, for example, an increase in heart rate, an increase in respiration rate or depth of respiration, or an increase in body temperature. In another example, the heart sound analyzer circuit 212 can trigger the HS sensing and feature generation only during a period when the detected metabolic demand is above a threshold. In an example, when the context detector 201 detects a time of day during which HS signal is acquired and analyzed, the heart sound analyzer circuit 212 can be configured to measure the plurality of measurements of a HS feature such as S3 heart sound strength (∥S3∥) only during specified time of a day, such as afternoon or a period of time excluding night time. In another example, the context detector 201 can detect a sleep or awake state of the patient, and the heart sound analyzer circuit 212 can perform multiple measurements of the ∥S3∥ only during the awake state. Details of the contextual information and the measurements of HS features based on the detected contextual information are discussed below, such as with reference to FIG. 4.

The cardiac electrogram sensor circuit 213 can be configured to sense from a patient at least one electrogram indicative of electrical activity of a heart. Examples of the electrogram can include intracardiac electrogram using one or more electrodes from the implantable leads 108A-C and the can 112, electrocardiogram (ECG) sensed using one or more surface electrodes placed on patient's skin, or bioelectrical signals indicative cardiac electrical activity that is sensed using subcutaneous electrodes disposed under the skin of the patient. The cardiac electrogram sensor circuit 213 can generate from the sensed electrogram one or more electrogram features such as a P wave, an R wave, a T wave, a QRS complex, or other components representing depolarization, hyperpolarization, repolarization, or other electrophysiological properties of the myocardium.

As an alternative or an addition to the cardiac electrograms, the physiologic signal sensing circuit 210 can include other sensor circuits configured to produce physiologic signals used for assisting the detection of a HS feature. For example, the physiologic signal sensing circuit 210 can be optionally coupled to one or more of a pressure sensor, an impedance sensor, an activity sensor, a temperature sensor, a respiration sensor, or a chemical sensor. These sensor, deployed to inside or otherwise associated with the patient body, can sense one or more physiological signals including heart rate, heart rate variability, electrocardiogram, intracardiac electrogram, arrhythmias, thoracic impedance, intracardiac impedance, arterial pressure, pulmonary artery pressure, left atrial pressure, RV pressure, LV coronary pressure, coronary blood temperature, physiologic response to activity, apnea hypopnea index, one or more respiration signals such as a respiration rate signal or a tidal volume signal, or blood oxygen saturation.

The target event indicator generator circuit 220 can be configured to generate a plurality of signal metrics from the one or more physiologic signals. The signal metric can include a statistical feature (e.g., mean, median, standard deviation, variance, correlation, covariance, or other statistical value over a specified time segment) or a morphological feature (e.g., peak, trough, slope, area under the curve). As illustrated in FIG. 2, the target event indicator generator circuit 220 can include a cardiac diastolic function detector circuit 221 configured to generate one or more signal metrics indicative of diastolic function of the heart, such as a S3 heart sound strength (∥S3∥). In some examples, the signal metrics can be composite signal metrics generated from two or more physiological signals.

The physiologic event detector circuit 230 can receive the signal metrics from the target event indicator generator circuit 220 and be configured to detect a physiologic target event or condition using the signal metrics such as representative of the relative timing between the first and second signal features. A target event or condition can include a physiologic event indicative of an onset of a disease, worsening of a disease state, or a change of a disease state. In an example, the physiologic event detector circuit 230 can detect the presence of an event indicative of HF decompensation status, change in HF status such as worsening of HF, pulmonary edema, or myocardial infarction. The physiologic event detector circuit 230 can be configured to generate a trend of representative values of the signal metrics over a specified time period, and to detect a target physiologic event using at least the trend of representative values of the signal metrics. In an example, the physiologic event detector circuit 230 can determine the trend by calculating a detection index (DI) representing the variation of the values of the signal metrics over time. For example, the DI can be computed as a difference between a first statistical measure of the signal metric computed from a first time window and a second statistical measure of the signal metric computed from a second time window. The first and the second statistical measures can each include a mean, a median, a mode, a percentile, a quartile, or other measures of central tendency of the signal metric values in the respective time window. In an example, the second time window can be longer than the first window, and at least a portion of the second time window precedes the first time window in time. The second statistical measure can represent a baseline value of the signal metric. In some examples, the signal metrics can be composite signal metrics generated from two or more physiological signals.

The controller circuit 240 can control the operations of the context detector 201, the physiologic signal receiver circuit 210, the target event indicator generator circuit 220, the physiologic event detector circuit 230, and the data flow and instructions between these components. The controller circuit 240 can receive external programming input from the instruction receiver circuit 250 to control one or more of detecting physiologic or environmental contextual conditions, sensing of physiologic signals, detecting contextual information, generating signal metrics, or detecting HF events. Examples of the instructions received by instruction receiver 250 may include: selection of electrodes or sensors used for sensing physiologic signals such as the electrograms and the heart sounds, detecting HS features representing the cardiac diastolic function, or the configuration of the HF event detection. The instruction receiver circuit 250 can include a user interface configured to present programming options to the user and receive user's programming input. In an example, at least a portion of the instruction receiver circuit 250, such as the user interface, can be implemented in the external system 120.

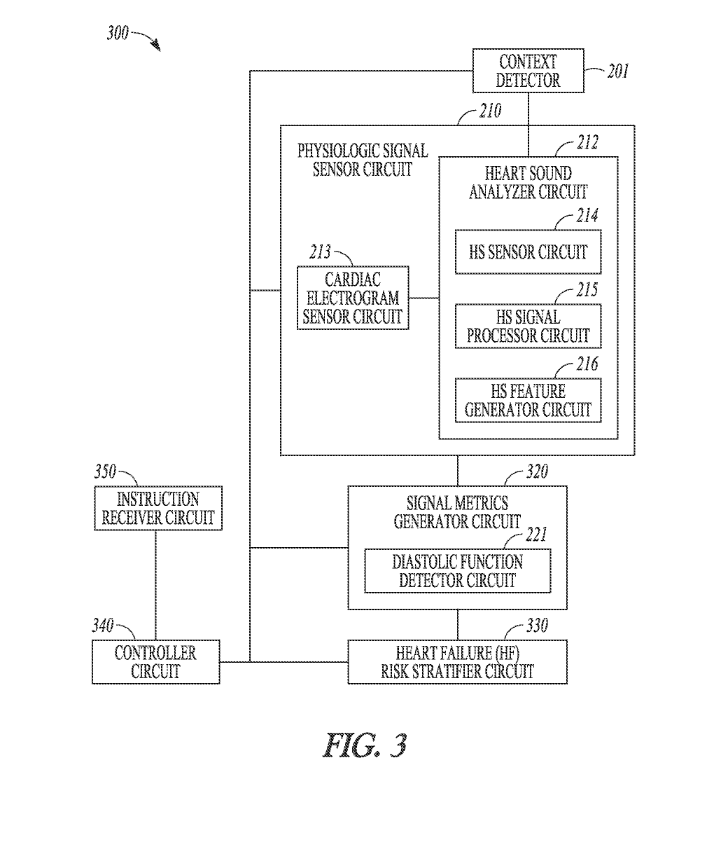

FIG. 3 illustrates an example of a heart sound-based heart failure risk stratifier circuit 300, which can be an embodiment of the heart sound-based HF event detection/risk assessment circuit 113. The heart sound-based heart failure risk stratifier circuit 300 can include one or more of a context detector 201, a physiologic signal receiver circuit 210, a signal metrics generator circuit 320, a heart failure (HF) risk stratifier circuit 330, a controller circuit 340, and an instruction receiver circuit 350.

As discussed in the heart sound-based physiologic event detector circuit 200 with reference to FIG. 2, the context detector 201 can be configured to detect contextual condition associated with a patient. The contextual condition can include a patient's physiologic context and an environmental context. The physiologic context includes body-related factor, such as the posture, activity level, sleep or awake state, mental or emotional state, metabolic demand, body temperature, and other parameters indicative of patient health status or body conditions. The environmental context can include factors external to the patient but likely to affect patient's health or disease states, such as the ambient temperature, barometric pressure, humidity, social environment, time of day, among others. The physiologic context or the environment context can correlate to or be indicative of a change in the patient's metabolic demand such as an elevated metabolic demand, medication intake schedule, among others. Details of the context detector 201 are discussed below, such as with reference to FIG. 4.

The physiologic signal receiver circuit 210 can receive one or more physiologic signals that can be indicative of worsening of HF status. The physiologic signal receiver circuit 210 can include a plurality of functional components including a heart sound analyzer circuit 212, and optionally a cardiac electrogram sensor circuit 213. The cardiac electrogram sensor circuit 213 and the heart sound analyzer circuit 212 can respectively sense from a physiologic sensor (e.g., the implantable leads 108A-C and the can 112, or implantable accelerometers or microphones), or retrieve from a database (e.g., an EMR system), or receive via an input device receive coupled to the instruction receiver 350, cardiac electrograms or heart sound signals. The cardiac electrogram sensor circuit 213 can process the sensed electrogram to produce the electrogram features (e.g., R waves or QRS complexes). The heart sound analyzer circuit 212, comprising the HS sensor circuit 214, HS signal processor circuit 215, and HS feature generator circuit 216, can be configured to process the HS signals such as filtering the HS using one or more filters, and produce and HS features (e.g., timing, amplitude, or morphology of S1, S2, S3 heart sounds). By receiving the contextual conditions from the context detector 201, the HS feature generator circuit 216 can perform a plurality of HS measurements of the one or more HS features in response to the detected contextual condition meeting a specified criterion.

The signal metrics generator circuit 320 can be configured to generate a plurality of signal metrics from the one or more physiologic signals. Examples of the signal metrics can include statistical features (e.g., mean, median, standard deviation, variance, correlation, covariance, or other statistical value over a specified time segment) and morphological features (e.g., peak, trough, slope, area under the curve). Similar to the target event indicator generator circuit 220 as illustrated in FIG. 2, the signal metrics generator circuit 320 can include a cardiac diastolic function detector circuit 221 configured to generate one or more signal metrics indicative of diastolic function of the heart, such as strength of S3 heart sound.

The heart failure (HF) risk stratifier circuit 330 can receive input from the signal metrics generator circuit 320, and calculate a composite risk indicator (CRI) using the one or more signal metrics such as produced by the signal metrics generator circuit 320. The CRI can indicate the likelihood of the patient developing a future event indicative of worsening of HF, such as developing a future HF decompensation event in a specified timeframe, such as within approximately 1-3 months, 3-6 months, or beyond 6 months. The HF risk stratifier circuit 330 can also be used to identify patients at elevated risk of developing a new or worsening of an existing disease, such as pulmonary edema, pulmonary condition exacerbation such as COPD, asthma and pneumonia, myocardial infarction, dilated cardiomyopathy (DCM), ischemic cardiomyopathy, systolic HF, diastolic HF, valvular disease, renal disease, chronic obstructive pulmonary disease (COPD), peripheral vascular disease, cerebrovascular disease, hepatic disease, diabetes, asthma, anemia, depression, pulmonary hypertension, sleep disordered breathing, hyperlipidemia, among others.

The controller circuit 340 can control the operations of the context detector 201, the physiologic signal receiver circuit 210, the signal metrics generator circuit 320, the HF risk stratifier circuit 330, and the data flow and instructions between these components. The controller circuit 340 can receive external programming input from the instruction receiver circuit 350 to control one or more of detecting physiologic or environmental contextual conditions, detecting the receiving cardiac electrogram, receiving heart sound signals, generating signal metrics including one or more signal metrics indicative of diastolic function of the heart, or calculating a composite risk. Examples of the instructions received by instruction receiver 350 may include: selection of electrodes or sensors used for sensing physiologic signals such as the electrograms and the heart sounds, selection of the signal metrics representing the cardiac diastolic function information, or the parameters used for calculating the composite risk. The instruction receiver circuit 350 can include a user interface configured to present programming options to the user and receive user's programming input. In an example, at least a portion of the instruction receiver circuit 350, such as the user interface, can be implemented in the external system 120.

FIG. 4 illustrates an example of a context receiver circuit 400, which can be an embodiment of the context detector 201. The context receiver circuit 400 can be configured to generate at least one contextual condition associated with a patient. When connected to the heart sound analyzer circuit 212, the context receiver circuit 400 can trigger a heart sound measurement session such that the heart sound analyzer circuit 212 can perform a plurality of HS measurements of the HS features in response to the at least one contextual condition meeting a specified criterion. The context receiver circuit 400 can also control the target event indicator generator circuit 220 or the signal metrics generator circuit 320 to generate one or more signal metrics using a portion of the measurements of the HS features, such as the measurements if and when at least one contextual condition meets a specified criterion.

The context receiver circuit 400 can include at least one of an environmental context detector 410 and a physiologic context detector 420. The environmental context detector 410 and the physiologic context detector 420 each can be coupled to a sensor that can sense a physical or physiological condition associated with the patient. In some examples, the environmental context detector 410 and the physiologic context detector 420 each can access from a machine-readable medium that stores the environmental context information or patient's medical record, or receive environmental context or the patient physiologic responses from an end-user such as via a user-interface connected to the instruction receiver circuit 250.

The environmental context detector 410 can be coupled a sensor that can sense a physical parameter indicative of the patient's ambient environmental condition external to the patient, but can likely affect the patient's health or disease states. The environmental context detector 410 can include one or more of a timer/clock circuit 411, an ambient temperature receiver 412, and a barometric pressure receiver 413.

The timer/clock circuit 411 can be capable of determining time of a day, such as morning, afternoon, or evening of the day. The heart sound analyzer circuit 212 can perform a plurality of HS measurements of the HS features during specified time of day when the patient is anticipated to have an elevated metabolic demand higher than other time of the day. In an example, the heart sound analyzer circuit 212 can measure HS during the afternoon of the day such as 4 p.m. to 6 p.m. In another example, the heart sound analyzer circuit 212 can measure HS during the period of the day excluding the night time (e.g., midnight to 6 a.m.).

The ambient temperature receiver 412 can receive values of the ambient temperature such as from a thermometer. The barometric pressure receiver 413 can receive values of the barometric pressure such as from a barometer. The ambient temperature receiver 412 or the barometric pressure receiver 413 can also respectfully receive the ambient temperature values or the barometric pressure values from an end-user input via the user-interface connected to the instruction receiver circuit 250. The ambient temperature or the barometric pressure may impact the patient's physiology and cause variations in signal metrics used for HF event detection or for HF risk stratification. For example, high environmental temperature or low barometric pressure can increase the metabolic demand of the patient, resulting in one or more presentations including an increase in heart rate, an increase in respiration rate or depth of respiration, an increase in body temperature, among other physiologic responses. These physiologic responses can further affect the diastolic function of the heart and cause fluctuation of the signal metrics such as S3 heart sound strength. The ambient temperature receiver 412 or the barometric pressure receiver 413 can trigger a HS measurement session, causing the HS feature generation circuit 216 to perform a plurality of HS measurements of the HS features when the ambient temperature or the barometric pressure meet respective criterion such as to correlate to an increase in the patient's metabolic demand. With the elevated metabolic demand, underlying HF disease or worsening of HF status can be more likely triggered and presented in the signal metrics such as S3 heart sound strength.

The physiologic context detector 420 can be coupled one or more sensors that can sense a parameter indicating patient's body condition, physiology, or psychological conditions, among others. The physiologic context detector 420 can include one or more of a metabolic demand detector 421, an anxiety level detector 422, a posture or activity detector 423, and a sleep/awake state detector 424. The metabolic demand detector 421 can be configured to detect the patient's metabolic demand, or the change of the metabolic demand. The metabolic demand detector 421 can be coupled to one or more physiologic sensors, including a heart rate sensor, a respiration sensor that can sense the change in respiration rate or tidal volume, a body temperature sensor that can sense a change in body temperature, or a chemical sensor that can sense oxygen or carbon dioxide level in the body. In an example, the metabolic demand detector 421 can detect a change in patient's metabolic demand when the one or more physiologic sensors produce information correlating to an increase in patient's metabolic demand, such as an increase in heart rate, an increase in respiration rate or depth of the respiration, or an increase in body temperature. The heart sound analyzer circuit 212, coupled to the context receiver circuit 400, can perform HS measurements in response to a detection of one or more of an increase in body temperature, an increase in heart rate, or an increase in respiration rate or depth of respiration.

The anxiety level detector 422 can detect an indication of the patient's anxiety or stress level during a specified period. The anxiety level detector 422 can be coupled to a physiologic sensor configured to detect an indication of patient stress level, or receive input about patient's stress level from an end-user input via the user-interface connected to the instruction receiver circuit 250. The heart sound analyzer circuit 212 can measure the plurality of HS measurements in response to a detection of a stress level correlating to or indicative of an increase in metabolic demand, such as when the detected stress level is above a specified threshold.

The posture or activity level detector 423 can be coupled a sensor such as an accelerometer and detect a posture of the patient as being one of two or more posture states including, for example, a supine or upright position. The posture or activity level sensor 423 can also detect patient's strenuousness of the activity. The heart sound analyzer circuit 212 can measure the plurality of HS measurements in response to the detected posture being classified as a specified state correlating to or indicative of the elevated metabolic demand, such as an upright posture. In an example, the heart sound analyzer circuit 212 can measure the plurality of HS measurements in response to a detection of an increase in the metabolic demand (such as produced by the metabolic demand detector 421 or the anxiety level detector 422) and the detected activity level below a specified threshold (such as reduced frequency, time, or vigorousness of activity over a given time period).

The sleep/awake state detector 424 can be configured to receive an indication of a change from a sleep state to an awake state, such as using a sleep detector. Examples of the sleep detector can include accelerometers, piezoelectric sensor, biopotential electrodes and sensors, or other physiologic sensors configurable to detect the posture, change of posture, activity, respiration, electroencephalograms, or other physiologic signals indicative of sleep or awake states. The sleep/awake state detector 424 can also receive indications of a sleep-to-awake state transition from an end-user such as via a user-interface connected to the instruction receiver circuit 250. In an example, the received transition from a sleep state to an awake state can be used to trigger a HS measurement sessions such that the HS feature generation circuit 216 can perform a plurality of HS measurements of the HS features upon the detection of a transition from a sleep to awake state.

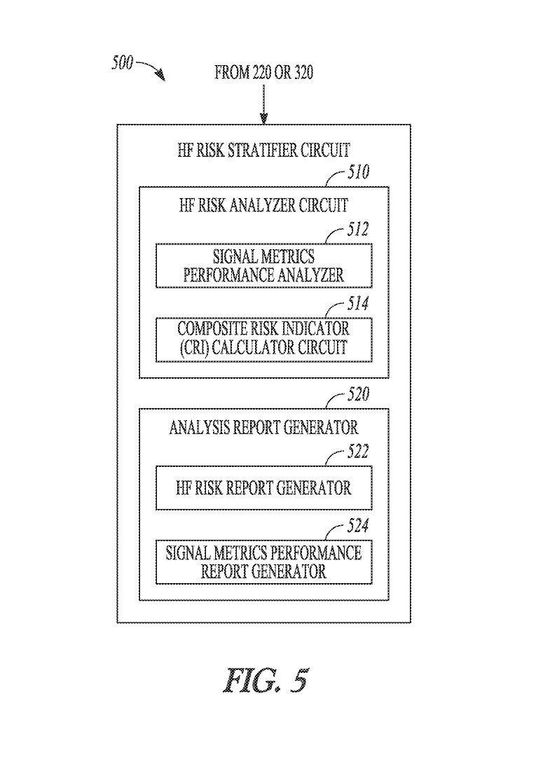

FIG. 5 illustrates an example of a HF risk stratifier/event detector circuit 500, which can be an embodiment of the HF risk stratifier circuit 330 or the physiologic event detector circuit 230. The HF risk stratifier/event detector circuit 500 can include an HF risk analyzer circuit 510 and an analysis report generator 520. The HF risk stratifier/event detector circuit 500 can receive the signal metrics indicative of cardiac diastolic function of the heart from the signal metrics generator circuit 320, analyze the signal metrics, and determine a quantity such as a composite risk indicator (CRI) indicative of the likelihood of the patient later developing a target physiologic event such as an HF decompensation event.

The HF risk analyzer circuit 510 can include a signal metrics performance analyzer 512 and a composite risk indicator (CRI) calculator circuit 514. The signal metrics performance analyzer 512 can be configured to generate for each of one or more of the signal metrics a respective performance measure that indicates reliability or accuracy of detecting a target physiologic event such as an HF decompensation event, or identifying patient at a higher risk of experiencing an HF decompensation event. Examples of the performance measures can include a predicted hazard ratio (HR), a predicted sensitivity (Se), a predicted specificity (Sp), or a predicted signal quality (Sq), each of which can be determined using population-based statistics. The signal metrics performance analyzer 512 can determine the predicted sensitivity of a signal metric such as S3 hear sound strength (∥S3∥) using the relative change of value of the signal metric in response to a physiologic status change associated with the progression of the HF status. In an example, the predicted sensitivity can be determined as a rate of change of the signal metric value from a first time to a second time, where the first and second time can be approximately 1-6 months and 14-28 days respectively prior to the patient's developing a target event such as an HF decompensation event.

The predicted specificity can characterize the accuracy of the signal metric in predicting a confounding event not associated with HF decompensation, such as noise, inference, patient activity, lead fracture, lead revision, change of pacing configuration, or a replacement of the device. The signal metrics performance analyzer 512 can determine the predicted specificity of a signal metric such as ∥S3∥ using the relative change of value of the signal metric from a first time to a second time in response to one or more confounding events. The first and second time can be approximately 1-6 months and 14-28 days respectively prior to patient's developing an HF decompensation event.

The signal metrics performance analyzer 512 can determine the predicted signal quality of a signal metric such as J∥S3∥. Examples of the signal quality can include signal strength, signal variability, or signal-to-noise ratio, among others. Signal variability can include range, inter-quartile range, standard deviation, variance, sample variance, or other first-order, second-order, or higher-order statistics representing the degree of variation. For example, in determining the quality of the signal metric of ∥S3∥, the signal metrics generator circuit 320 can produce a plurality of HS measurements of ∥S3∥ such as from a plurality of cardiac cycles during a specified period of time. The signal metrics performance analyzer 512 can determine the variability of the ∥S3∥ measurements such as by computing a variance of the ∥S3∥ measurements. A high signal quality, such as indicated by one or more of high signal-to-noise ratio, high signal strength, or low signal variability, is desirable for identifying patients at an elevated risk of developing future HF events.

The composite risk indicator (CRI) calculator circuit 514 can generate a CRI using one or more signal metrics. In an example, the signal metrics performance analyzer 512 can calculate for each signal metrics (Mᵢ) a respective individual risk score (R_{Mi}) using a probability model (f) and one or more of the predicted hazard ratio (HR), the predicted sensitivity (Se), the predicted specificity (Sp), and the predicted signal quality (Sq). That is, R_{Mi}=f(HR, Se, Sp, Sq). The CRI calculator circuit 514 can compute the CRI using a linear or nonlinear combination of the risk scores (R_{Mi}) associated with respective signal metrics. The CRI can be computed as weighted sum of the risk scores, where each risk score can be scaled by a respective weight factor proportional to a performance measure of the signal metric. The CRI can also be determined as a parametric or non-parametric model using the individual risk scores, such as decision trees, neural network, Bayesian network, among other machine learning methods.

The analysis report generator 520 can include an HF risk report generator 522 and a signal metrics performance report generator 524. The HF risk report generator 522 can generate a report to inform, warn, or alert a system end-user an elevated risk of a patient developing a future HF event. The report can include the CRI with corresponding timeframe within which the risk is predicted. The report can also include recommended actions such as confirmative testing, diagnosis, or therapy options. The report can include one or more media formats including, for example, a textual or graphical message, a sound, an image, or a combination thereof. In an example, the HF risk report generator 522 can be coupled to the instruction receiver circuit 250 and the report can be presented to the user via an interactive user interface on the instruction receiver circuit 250. The HF risk report generator 402 can be coupled to the external device 120, and be configured to present to the user the risk (e.g., the CRI) of patient developing future HF events via the external device 120.

The signal metrics performance report generator 524 can generate, and present to the user, one or more of a report including the contextual conditions such as detected by the context detector 201, the cardiac electrograms such as received by the cardiac electrogram receiver circuit 313, the heart sound signals such as received by the heart sound analyzer circuit 212, and the signal metrics representing the cardiac diastolic function information such as generated by the signal metrics generator circuit 320. The signal metrics performance report generator 524 can be coupled to the external device 120 or the instruction receiver circuit 250, and be configured to present the signal metrics information to the user therein. The user input can include confirmation, storage, or other programming instructions to operate on the signal metrics.

FIG. 6 illustrates examples of receiver operating characteristics (ROC) curves corresponding to representative S3 heart sound strength (∥S3∥) calculated from heart sound signals acquired during different time of day. The heart sound can be sensed using an implantable accelerometer disposed within an implantable medical device. A ∥S3∥ trend can be determined as difference between a first statistical measure from a plurality of ∥S3∥ measurements within a first time window and a second statistical measure from a plurality of ∥S3∥ measurements within a second time window. The first and second statistical measures each can include a mean, a median, a mode, a percentile, a quartile, or other measures of central tendency of the signal metric values in the respective time window. In an example, the second time window can be longer than the first window, and at least a portion of the second time window precedes the first time window in time. The second statistical measure can be indicative of a baseline ∥S3∥ value.

The ROC curves can be used to illustrate and evaluate the performance of a detector or a detection algorithm in detecting the target events indicative of HF decompensation. The ROC curves depict the sensitivities of detecting the target event (as shown in the y-axis) over the corresponding patient-year false alarm rates (as shown in the x-axis) for a plurality of detection thresholds, such as the threshold for the ∥S3∥ trend of the difference between the statistical measures calculated from the short-term window and the statistical measures calculated from the long-term window.

The ROC curves 601, 602 and 603 respectively correspond to ∥S3∥ detected and measured from heart sound signals sensed during the day time (approximately 12 p.m. to 4 p.m. during a day), during the night time (approximately 12 a.m. to 4 a.m. the next day), and during a 24-hour period. As illustrated in FIG. 6, for a specified false alarm rate selected from a wide range of false positive rate, a higher sensitivity can be achieved from the ROC curves 601 and 603 than from the ROC curve 602. The area under the ROC curve (A_{ROC}), an index that can be used to evaluate a detector's performance, can be computed for each of the ROC curves 601, 602 and 603. A qualitative comparison indicates that the A_{ROC} of 601 and the A_{ROC} of 603 are larger than the A_{ROC} of 602. Therefore, in the context of detecting events indicative of HF decompensation, the example shown in FIG. 6 suggests that the representative ∥S3∥ detected and measured from the heart sound signals acquired during the day time or during a 24-hour period outperforms the representative ∥S3∥ detected and measured from the heart sound signals acquired during the night time.

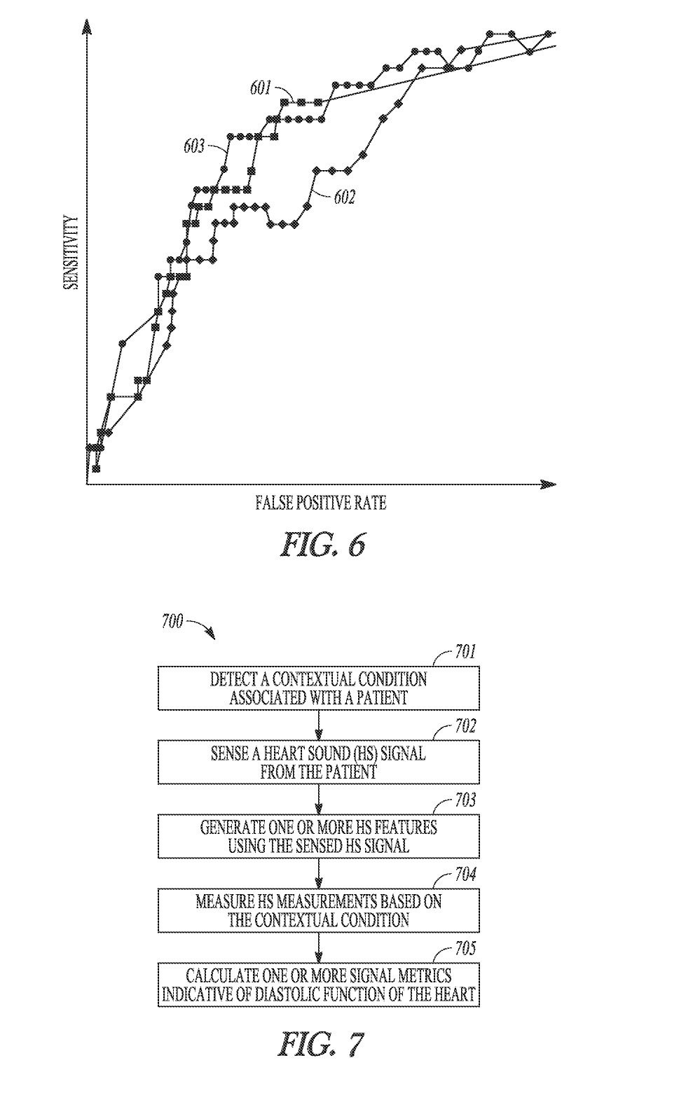

FIG. 7 illustrates an example of a method 700 for evaluating cardiac diastolic function of a patient. The method 700 can be implemented and operate in an ambulatory medical device or in a remote patient management system. In an example, the method 700 can be performed by the heart sound-based HF event detection/risk assessment circuit 113 implemented in the IMD 110, or the external device 120 which can be in communication with the IMD 110.

At 701, a contextual condition associated with a patient can be detected. The contextual condition can include at least one of a patient's physiologic context or an environmental context. The physiologic context can include body-related contextual information, such as a posture, an activity level, a sleep or awake state, a mental or emotional state, metabolic demand, body temperature, and other parameters indicative of patient health status or body conditions. The environmental context can include factors external to the patient but likely to affect patient's health or disease states, such as the ambient temperature, barometric pressure, humidity, social environment, among others. The physiologic context or the environment context can correlate to the patient's elevated metabolic demand. The contextual condition can be sensed using a physical sensor or a physiologic sensor. For example, a timer/clock circuit can be used to provide contextual condition of time of a day, a thermometer can provide ambient temperature, a body temperature sensor can provide an indication of a change of the patient's metabolic demand, an accelerometer can provide information regarding one or more of patient's posture, activity level, or indication of the sleep/awake state. Alternatively or additionally, the contextual condition can be obtained via a user-input such as via the user-interface connected to the instruction receiver circuit 250.

At 702, a physiologic signal indicative of heart sound (HS) of the patient can be received. The signal indicative of HS can be sensed using a HS sensor that can detect heart sound wave or other forms of signals such as vibrations of the chest wall resulting from cardiac mechanical contraction and relaxation. Examples of the HS sensors can include an ambulatory accelerometer or an ambulatory microphone. The physiologic signals indicative of HS can be stored in a storage device such as an EMR system, and can be retrieved from the storage device upon receiving a command such as issued by an end-user.

At 703, the received HS signal can be processed, including amplification, digitization, filtering, or other signal conditioning operations, and one or more HS features can be generated from the processed HS signal. Examples of the HS features can include timing, amplitude, or morphological features of S1, S2 or S3 heart sounds. In an example, the HS features can be generated from one or more time windows of the HS signal with reference to a physiologic event such as R waves or QRS complexes from an electrogram. For example, an S1 detection window can begin at 50 milliseconds (msec) following a detected R wave and have a duration of 300 msec. An S2 detection window can begin at specified offset following a detected R wave or S1 heart sound. An S3 detection window can be determined using at least one cardiac signal feature such as the R-wave timing or the timing of S2 heart sound. The S3 detection window can have a specified duration and can begin at a specified offset following the detected S2. In an example, the offset can be 125 msec, and the S3 window duration can be 125 msec. The offset or the S3 window duration can be a function of a physiologic variable such as a heart rate. For example, the offset can be inversely proportional to the heart rate, such that the S3 detection window can start at a smaller offset following the S2 at a higher heart rate.

A portion of the HS signals within the respective HS detection window can be used to detect the HS features. For example, the HS feature includes an S3 heart sound strength (∥S3∥) that can be determined as the HS signal energy (E_{S3Win}) within the S3 detection window. An S3 is detected if E_{S3Win} exceeds a specified threshold. In an example, the HS features can be detected adaptively by tracking the temporal locations of the previously detected HS features. For example, an S3 heart sound can be detected by adaptively tracking the timing of historically detected S3 heart sounds. A dynamic programming algorithm can be used to detect and track the S3 heart sound within the S3 detection window, such as that disclosed in the commonly assigned Patangay et al. U.S. Pat. No. 7,853,327 entitled “HEART SOUND TRACKING SYSTEM AND METHOD,” which is hereby incorporated by reference in its entirety. For the detected HS feature such as the S3 heart sound, the S3 strength ∥S3∥ can be determined as the amplitude of the S3 heart sound in the time-domain HS signal, a transformed HS signal such as integrated HS energy signal), or in a frequency-domain HS signal such as the peak value of the power spectral density. In some examples, the S3 strength ∥S3∥ can be determined as the peak value of a generic measurement within the S3 detection window, such as peak envelop signal or root-mean-squared value of the portion of the HS signal within the S3 detection window.

At 704, a plurality of HS measurements of the one or more HS features can be measured in response to the detected patient contextual condition meeting a specified criterion. In an example where the detected contextual condition includes a timer/clock, a plurality of HS measurements ∥S3∥ can be measured during specified time of a day, such as in evenings from approximately 4:00 p.m. to 6 p.m. In another example where the detected contextual condition includes the patient's sleep or awake state, multiple measurements of ∥S3∥ can be performed only during a specified state, such as when the patient is awake. In yet another example where the detected contextual condition includes an indication of patient's metabolic demand or sleep/awake state, multiple measurements of ∥S3∥ can be performed when the metabolic demand exceeds a specified level such as the patient respiration rate or the body temperature exceeding respective threshold, or when the patient is awake. In various examples, HS measurements can be performed only when one or more contextual conditions correlate to or indicate an elevated metabolic demand in the patient, such as an increase in heart rate, an increase in respiration rate or depth of respiration, or an increase in body temperature.

At 705, one or more signal metrics can be calculated using at least the HS measurements. The signal metric can include a statistical feature (e.g., mean, median, standard deviation, variance, correlation, covariance, or other statistical value over a specified time segment) or a morphological feature (e.g., peak, trough, slope, area under the curve). The signal metrics can include parameters indicative of diastolic function of the heart, such as a ∥S3∥. In some examples, the signal metrics can be composite signal metrics generated from two or more physiological signals. The signal metrics can be presented to the end-user for monitoring the patient health status or disease progress such as worsening of HF. The signal metrics can also be used for detecting the presence of a target physiologic event such as an indication of an HF decompensation event, for predicting the future risk of developing a target physiologic event, or for titrating medical or device therapies to the patient such as by adjusting the dosage or parameters associated with the electrical stimulation.

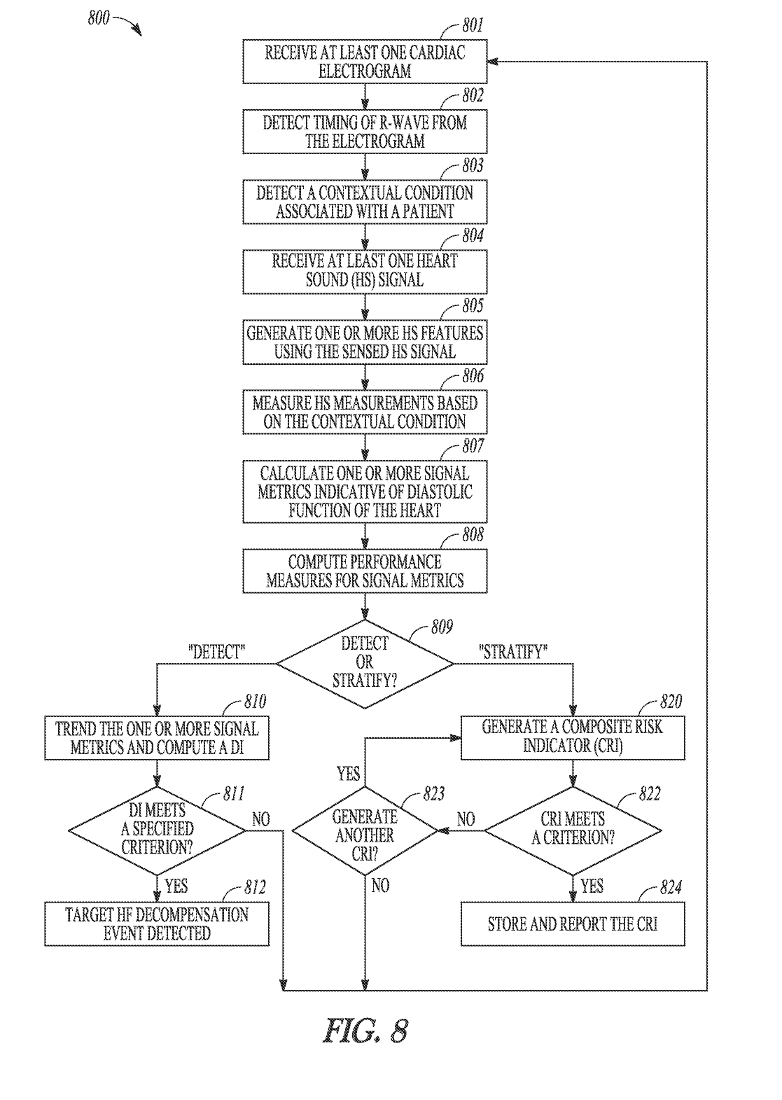

FIG. 8 illustrates an example of a method 800 for detecting an HF event indicative of HF decompensation or providing a risk stratification of a future HF event. The method 800 can be an embodiment of the method 700 for evaluating cardiac diastolic function of a patient, further including method for detecting a present HF event or predicting a future HF event using the cardiac diastolic function evaluation. In an example, the method 700 can be performed by the HS-based HF event detection/risk stratification circuit 113.

At 801, at least one cardiac electrogram can be received from a patient. The cardiac electrogram can include intracardiac electrogram, surface electrocardiogram (ECG), subcutaneous electrogram, or any other cardiac signals indicative of electrical activity of a heart. The cardiac electrogram can be sensed using a physiologic sensor or a plurality of electrodes attached to or implanted within the patient body, such as two or more electrodes on one or more of transvenous leads 108A-C coupled to an implantable medical device (IMD) and the can 112 of the IMD. The cardiac electrogram can be retrieved from a database such as residing in an electrical medical record (EMR) system that retrievably stores patient's electrograms. The received cardiac electrogram can be analyzed at 802 to generate one or more electrogram features including a P wave, R wave, T wave, QRS complex, or other components representing depolarization, hyperpolarization, repolarization, or other electrophysiologic properties of the myocardium.

At 803, a contextual condition, including one or more of a patient's physiologic contexts or environmental contexts associated with the patient, can be detected such as using one or more physical sensors or physiologic sensors. At least one heart sound (HS) signal can be sensed at 804 using a heart sound sensor. At 805, one or more HS features, such as timing, amplitude, or morphologic features of one or more of S1, S2, and S3 heart sounds, can be generated from the sensed HS signal at 805. At 806, a plurality of HS measurements of the one or more HS features can be measured in response to the detected patient contextual condition meeting a specified criterion. In various examples, the HS measurements can be performed if and when the measurement is performed in a specified period of time during a day such as afternoon; when the patient is awake, when the body temperature, the respiration rate, or the stress level is within a specified range; or when the air temperature or the barometric pressure in the patient's ambient environment is within a specified range.

At 807, one or more signal metrics can be calculated using at least the HS measurements, such as by computing a statistical measure out of the HS measurements. Using at least the HS measurements, performance measures can be computed for one or more signal metrics at 808. The performance measure of a signal metric can include a predicted hazard ratio (HR), a predicted sensitivity (Se), a predicted specificity (Sp), or a predicted signal quality (Sq) determined using population-based statistics. The predicted sensitivity of a signal metric, such as ∥S3∥, can include a relative change of value of the signal metric in response to a physiologic status change associated with the progression of the HF status. The predicted specificity of a signal metric can include a relative change of value of the signal metric from a first time to a second time in response to one or more confounding events not associated with HF decompensation. Examples of the confounding events can include noise, inference, patient activity, lead fracture, lead revision, change of pacing configuration, or a replacement of the device. The predicted signal quality of a signal metric, such as ∥S3∥, can include signal strength, signal variability, or signal-to-noise ratio, among others. The performance measure of a signal metric can be computed using a probability model and one or more of the predicted hazard ratio (HR), the predicted sensitivity (Se), the predicted specificity (Sp), and the predicted signal quality (Sq).

At 809, a decision is made, such as by an end-user through a programming device, to select either using the calculated performance measurement to detect the presence of an event indicative of HF decompensation, or to stratify patient's risk of developing a future event indicative of HF decompensation. If the choice is to “detect” HF decompensation, then at 810, a trend can be generated using the one or more signal metrics, such as the ∥S3∥. The trend of a signal metric can indicate variation (such as increase or decrease) of the signal metric value over time. The trend can be quantitatively represented by a detection index (DI). The DI can be calculated as a difference between a first statistical measure of the signal metric computed from a first time window and a second statistical measure of the signal metric computed from a second time window. The first and the second statistical measures can each include a mean, a median, a mode, a percentile, a quartile, or other measures of central tendency of the signal metric values in the respective time window. In an example, the second time window can be longer than the first window, and at least a portion of the second time window precedes the first time window in time. The second statistical measure can be indicative of a baseline value. For example, a DI from the trend of ∥S3∥ can be the difference between an average ∥S3∥_{W1} in the first window and an average ∥S3∥ in the second window representing the baseline ∥S3∥ (∥S3∥_{Baseline}), that is, DI=∥S3∥_{W1}−∥S3∥_{Baseline}. In another example, the DI can be computed as a rate of change from the second statistical measure to the first statistical measure. For example, the DI for the ∥S3∥ can be determined as (∥S3∥_{W1}−∥S3∥_{Baseline})/(T_{W1}−T_{Baseline}), where T_{Baseline} and T_{W1} are the representative time for the first and second time window, respectively.

A decision is made at 811 as to whether the DI meets a specified criterion, such as exceeding a specified threshold. The target HF decompensation event is deemed detected at 812 if, for example, the increase or the rate of increase in ∥S3∥ exceeds a specified threshold. If the DI does not meet the criterion, then no target HF event is deemed detected, and the patient monitoring can be continued with receiving the physiological signals such as the cardiac electrogram at 801.

If at 809 the choice is made to “stratify” the patient's risk, then at 820 a composite risk indicator (CRI) can be generated. The CRI can be a quantity that indicates the likelihood of the patient developing a future event indicative of worsening of HF, such as excessive intrathoracic fluid accumulation, increased heart sounds, increased heart rate, increased respiratory rate, decreased tidal volume, reduction in activity, or other events indicative of HF decompensation status. The CRI can be calculated using one or more signal metrics indicative of cardiac diastolic function information. In an example, the CRI can be computed as a linear or nonlinear combination of individual risk scores associated with respective signal metrics. The CRI can also be determined as a parametric or non-parametric model using the individual risk scores, such as decision trees, neural network, Bayesian network, among other machine learning methods.

At 822, the CRI is checked against a specified criterion, such as a reference or threshold value, to determine the risk of the patient developing a future HF event. The reference measure can be computed from data from a patient population. The reference measure computed from such a population can indicate an “average” patient's risk of developing future HF events. The reference measures can include: the mean, median, a range, or other central tendency of the risk across the patient population; variance, standard deviation, or other second or higher order statistical measures across the patient population; histogram, statistical distribution, or the parametric or non-parametric model representing the histogram or statistical distributions.

The comparison can include computing dissimilarity between the CRI and the reference. Examples of the dissimilarity can include a difference, a ratio, a percentile change, or other relative change. The dissimilarity can be computed as multi-dimensional distance using the statistical distribution of the reference measure. The dissimilarity can be compared to one or more thresholds such that the CRI can be categorized to two or more categorical risk levels indicating elevated risk of the patient later developing the HF event. For example, the categorical levels can include “high risk”, “medium risk”, or “low risk.” A higher degree of dissimilarity between the CRI and the reference can indicate a higher risk of the patient developing HF events in the future than an average patient with the similar chronic conditions.

If at 822 the CRI meets the specified criterion such as the CRI value being categorized as “medium risk” or “high risk” level, then at 824 a report is generated to inform, warn, or alert the user the elevated risk of patient's developing a future HF event. The report can include any or all of the information of the signal metrics selected for analysis, the CRI, the categorical classifications of CRI, one or more composite risk indices with corresponding timeframe within which the risk is predicted. The report can also include recommendations for intervention, further testing, or treatment options for the patient. The report can be in a form of a textual or graphical message, a sound, an image, or any combination thereof.

If the CRI does not meet the specified criterion, then at 823 a decision is made as to whether a new CRI is to be computed such as using additional signal metrics. The decision at 823 can be received from an end-user such as using a programming device, or automatically executed in response to the CRI failing to meet the criterion by a narrow margin. If additional signal metrics are decided to be used at 823, then a new CRI can be generated at 821; otherwise, the patient is deemed at low risk of developing future HF event, and no preventive action is deemed necessary. The patient monitoring can be continued with receiving the physiological signals such as the cardiac electrogram at 801.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

What is claimed is:

1. A system, comprising:
an ambulatory medical device (AMD) including:
a context detector circuit configured to detect contextual condition associated with a patient, the contextual condition including information correlating to or indicative of a change in metabolic demand of a patient;
a heart sound analyzer circuit configured to sense from the patient a vibratory or acoustic heart sound (HS) signal, generate one or more HS features using the sensed HS signal, and perform a plurality of HS measurements of the one or more HS features in response to the detected patient contextual condition meeting a specified criterion;
a target event indicator generator circuit configured to calculate one or more signal metrics indicative of diastolic function of a heart of the patient using the plurality of HS measurements, the signal metrics including a trend of the one or more HS features; and
a physiologic event detector circuit coupled to the target event indicator generator circuit, the physiologic event detector circuit configured to detect a target physiologic event using the one or more signal metrics.

2. The system of claim 1, wherein the target event indicator generator circuit is configured to calculate the one or more signal metrics including a trend of third (S3) heard sound, and wherein the physiologic event detector circuit is configured to detect the worsening of heart failure using the trend of S3 heart sound.

3. The system of claim 1, comprising a cardiac signal sensor and an HS feature detection window generator coupled to the heart sound analyzer circuit, wherein:
the cardiac signal sensor is configured to sense a physiologic signal indicative of electrical activity of the heart and to generate one or more cardiac signal features using the sensed physiologic signal; and
the HS feature detection window generator is configured to generate one or more HS feature detection windows using the one or more cardiac signals features;
and wherein the heart sound analyzer circuit configured to detect the S3 heart sound within the one or more HS feature detection windows.

4. The system of claim 3, wherein the HS feature detection window generator is configured to generate an S3 heart sound detection window using at least one cardiac signal feature including an R-wave timing or at least one HS feature including timing of S2 heart sound.

5. The system of claim 1, wherein the heart sound analyzer circuit is configured to detect the S3 heart sound by adaptively tracking timing of historically detected S3 heart sounds.

6. The system of claim 1, wherein the context detector circuit includes a timer/clock circuit capable of determining time of a day, and wherein the heart sound analyzer circuit is configured to measure the plurality of HS measurements during specified time of a day correlating to or indicative of an elevated metabolic demand of the patient.

7. The system of claim 6, wherein the heart sound analyzer circuit is configured to measure the plurality of HS measurements during a period of time excluding night of the day.

8. The system of claim 1, wherein the context detector circuit includes a sleep state detector configured to detect in the patient a time of transition from a sleep state to an awake state, and wherein the heart sound analyzer circuit is configured to measure the plurality of HS measurements in response to the detected transition from the sleep state to the awake state.

9. The system of claim 1, wherein the context detector circuit includes a posture sensor configured to detect a posture of the patient and to classify the posture as one of two or more posture states, and wherein the heart sound analyzer circuit is configured to measure the plurality of HS measurements in response to the detected posture being classified as a specified state correlating to or indicative of an elevated metabolic demand.

10. The system of claim 1, wherein the context detector circuit includes one or more physiologic sensors configured to detect a change in metabolic demand of the patient during a specified period, and wherein the heart sound analyzer circuit is configured to measure the plurality of HS measurements in response to a detection of an increase in the metabolic demand.

11. The system of claim 10, wherein the physiologic sensor includes one or more of a body temperature sensor, a heart rate sensor, a pressure sensor, or a respiration sensor, and the heart sound analyzer circuit is configured to measure the plurality of HS measurements in response to a detection of one or more of an increase in body temperature, an increase in heart rate, an increase in pressure, or an increase in respiration rate.

12. The system of claim 11, further comprising an activity sensor configured to detect activity level of the patient, wherein the heart sound analyzer circuit is configured to measure the plurality of HS measurements in response to a detection of an increase in the metabolic demand and the detected activity level below a specified threshold.

13. A system, comprising:
a signal analyzer circuit, including:
a context detector circuit configured to receive contextual condition associated with a patient, the contextual condition including information correlating to or indicative of a change in the metabolic demand of a patient;
a heart sound analyzer circuit configured to receive a vibratory or acoustic heart sound (HS) signal of the patient, generate one or more HS features using the sensed HS signal, and measure a plurality of HS measurements of the one or more HS features in response to the received patient contextual condition meeting a specified criterion; and
a signal metrics generator circuit configured to calculate one or more signal metrics indicative of diastolic function of a heart of the patient using the plurality of HS measurements, the signal metrics including a trend of the one or more HS features; and
a risk stratifier circuit configured to generate a composite risk indicator using the one or more signal metrics, the composite risk indicator indicative of the likelihood of the patient developing a future event indicative of a new or worsening of an existing disease.

14. The system of claim 13, further comprising a cardiac signal sensor and a HS feature detection window generator coupled to the heart sound analyzer circuit, wherein:
the cardiac signal sensor is configured to sense a physiologic signal indicative of cardiac electrical activity and to generate one or more cardiac signal features using the sensed physiologic signal; and
the HS feature detection window generator is configured to generate one or more HS feature detection windows using the one or more cardiac signals features, the one or more HS feature detection windows including an S3 detection window;
and wherein the heart sound analyzer circuit is configured to detect an S3 heart sound within the S3 detection window.

15. The system of claim 13, wherein the risk stratifier circuit is configured to generate two or more categorical risk levels using a comparison between the composite risk indicator and a reference measure, the two or more categorical risk levels indicative of elevated risk of the patient developing a future event indicative of worsening of heart failure.

16. A method, comprising:
detecting a contextual condition associated with a patient, the contextual condition including information correlating to or indicative of elevated metabolic demand of a patient;
sensing from the patient a heart sound (HS) signal;
generating one or more HS features using the sensed HS signal;
measuring a plurality of HS measurements of the one or more HS features in response to the detected patient contextual condition meeting a specified criterion; and
calculating one or more signal metrics indicative of diastolic function of the heart using the plurality of HS measurements, the signal metrics including a trend of the one or more HS features.

17. The method of claim 16, wherein detecting the contextual condition includes detecting time of a day, and wherein measuring the plurality of HS measurements includes measuring a plurality of HS measurements of S3 heart sound in response to the detected time of the day correlating to or indicative of elevated metabolic demand.

18. The method of claim 16, wherein detecting the contextual condition includes, using one or more physiologic sensors, detecting at least one of a time of transition from a sleep state to an awake state, an increase in body temperature, an increase in heart rate, an increase in pressure, a decrease in activity level, or an increase in respiration rate.

19. The method of claim 16, comprising:
sensing a physiologic signal indicative of cardiac electrical activity; and
generating one or more cardiac signal features using the sensed physiologic signal;
and wherein generating the one or more HS features includes generating S3 heart sound using the one or more cardiac signals features and the sensed HS signal.

20. The method of claim 16, further comprising at least one of:
detecting a target physiologic event indicative of worsening of heart failure using the one or more signal metrics; or
generating a composite risk indicator using the selected one or more signal metrics and classifying the patient into one of two or more categorical risk levels, the composite risk indicator indicative of the likelihood of the patient developing a future event indicative of worsening of heart failure.
